(19) United States
(12) Patent Application Publication
(10) Pub. No.: US 2006/0265052 A1
(43) Pub. Date: Nov. 23, 2006

(54) Deployable stent

(75) Inventor: Zhong You, Oxford (GB)

Correspondence Address: NIXON & VANDERHYE, PC, 901 NORTH GLEBE ROAD, 11TH FLOOR, ARLINGTON, VA, 22203, US

(73) Assignee: ISIS INNOVATION LIMITED, Summerton (GB)

(21) Appl. No.: 11/438,706

(22) Filed: May 23, 2006

Related U.S. Application Data
(63) Continuation-in-part of application No. 10/473,232, filed on Dec. 16, 2003, now Pat. No. 7,060,092.

(30) Foreign Application Priority Data
Mar. 29, 2001 (GB) 0107910.2

(51) Int. Cl. A61F 2/88; A61F 2/90

(52) U.S. Cl. 623/1.22; 623/1.15

(57) ABSTRACT

A stent 1 comprises a sheet 2 of biocompatible material having a tubular shape and folded with a pattern of folds allowing the sheet 2 to be collapsed for deployment. The pattern of folds comprises a unit cell repeated over the sheet 2. The unit cell comprises: two longitudinal folds extending away from a common point along the tubular shape of the sheet, the first longitudinal fold being of the first type and the second longitudinal fold being of the second type; an outer circumferential ring of four edge folds of the first type, comprising, on each side of the longitudinal folds, a minor edge fold extending from the outer end of the first longitudinal fold and a major edge fold extending from the outer end of the second longitudinal fold, the outer ends of the minor edge fold and the major edge fold on the same side of the longitudinal folds intersecting one another; and two angular folds of the second type, each extending from the intersection of a major edge fold with a minor edge fold to the common point from which the longitudinal folds extend. The stent 1 prevents tissue in-growth because it is formed of a continuous sheet 2.

### CROSS-REFERENCE TO RELATED APPLICATION

This application is a Continuation-In-Part of co-pending application Ser. No. 10/473,232 which is itself the US national phase of International Patent Application No. PCT/GB02/01424, filed 27 Mar. 2002.

### BACKGROUND OF THE INVENTION

(1) Field of the Invention

The present invention relates to a stent. The present invention provides a novel structure for a stent.

A stent is a medical device designed to open up a blocked lumen at a site in the human (or even animal) body, for instance a coronary artery, or the oesophagus etc., or used to protect a damaged or weakened vessel such as an aorta. An occlusion might be caused for instance by a disease such as stenosis or by cancer. A weakening of a blood vessel may be caused by an aneurism. Stents preferably have a flexible structure allowing them to be collapsed to reduce their outer dimensions. This is to facilitate the passage of the stent into the site in the body where the stent is expanded for deployment. Typical uses of a stent are to open blocked coronary arteries and large veins, to treat obstructions to breathing in the trachea and bronchus, to allow the passage of urine in the prostate and to palliate cancer stenosis in the oesophagus. More recently, it is regarded as a beneficial treatment for an Abdominal Aortic Aneurism. Stent therapy is now widely accepted for interventional treatment not only in the vascular system, but also the gastrointestinal, belier and urinary systems. Stent techniques have come to be regarded as simply, safe and effective in comparison to other surgical or non-surgical treatments.

(2) Description of the Related Art

Known stents have one of five basic constructions that is tubular, coil, ring, multi-design and mesh structures. Tubular stents are rigid. The other types of known structures are collapsible and typically comprise an open tubular structure of structural elements which may be collapsed to facilitate deployment. The various known structures have different features and advantages, for example high expansion rate, suitable stiffness, good flexibility and/or good tractability. Whilst some structures provide different combinations of these advantages, an ideal stent sharing all these advantages has yet to be realised.

One of the major problems with known stents is restenosis occurring after implantation. This is a particular problem for mesh stents and other open structures as tissues grow through the stent and block the lumen again and is a particular problem in oesophageal applications. Some reports suggest that restenosis is due to cell damage occurring during deployment at the blocked site as the stent pushes against the cell wall. The amount of such damage is dependent on the stent configuration. After significant tissue growth through a stent, the stent cannot be retrieved. Thus it may be necessary to implant further stents after a first stent becomes blocked in order to reopen the blockage. As this involves stents being implanted inside one another, there is a limit to the number of stents which can be implanted at one location.

To overcome this problem, covered stents have been developed. Covered stents were developed by attaching a tubular flexible cover, for example of polyester, attached around the outside of a wire mesh stent structure. The use of such a cover around a wire mesh stent is an effective way to prevent tissue in-growth. Moreover, for other diseases such as an Abdominal Aortic Aneurism, covered stents are necessary to isolate aneurisms. However, the common problems of covered stents include a risk of rupture of the cover, migration/slippage of the stent, and difficulties in delivery due to the large packaged size. The risk of slippage and hence migration of the stent is a particular problem. Such covered stents still rely, for example, on a mesh frame for collapse and expansion during deployment, but there has been very little investigation of the integrated expanding mechanism when the stent is covered.

As a result of the problems described above for both covered and uncovered stents re-intervention is often required. As a result many patients have sub-optimal response to this type of treatment.

Current expandable stents are expensive to manufacture due to their complicated structures which are labourious to form. The high cost has reduced their widespread use.

The present invention is intended to provide a stent which avoids at least some of the problems discussed above.

### BRIEF SUMMARY OF THE INVENTION

According to the present invention, there is provided a stent comprising a biocompatible sheet having a tubular shape and being folded with a pattern of folds allowing the sheet to be collapsed for deployment of the stent, the folds being of two types, the first type being one of a hill fold and a valley fold, and the second type being the other of a hill fold and a valley fold, the pattern of folds comprising a unit cell repeated over at least a portion of the sheet, the unit cell comprising:

two longitudinal folds extending away from a common point along the tubular shape of the sheet, the first longitudinal fold being of the first type and the second longitudinal fold being of the second type;

an outer circumferential ring of four edge folds of the first type, comprising, on each side of the longitudinal folds, a minor edge fold extending from the outer end of the first longitudinal fold and a major edge fold extending from the outer end of the second longitudinal fold, the outer ends of the minor edge fold and the major edge fold on the same side of the longitudinal folds intersecting one another; and

two angular folds of the second type, each extending from the intersection of a major edge fold with a minor edge fold to the common point from which the longitudinal folds extend.

Such a structure for a stent provides numerous advantages.

As the stent comprises a sheet, tissue in-growth is prevented or isolation of aneurisms is possible. Furthermore, the pattern of folds allows the sheet to be collapsed for deployment facilitating delivery to the blocked site in the body. The pattern of folds allows the sheet to be collapsed radially of the tubular shape. The use of a pattern of folds to collapse the stent allows it to be packaged compactly and to have good flexibility for ease of delivery to the blocked site. The structure can be simple in structural form and is hingeless which increases reliability. The pattern of folds also provides for synchronised deployment across the sheet which reduces the chances of rupture on deployment. The ability to fold the sheet compactly allows the use of relatively strong materials which would otherwise not be deployable. Such strong materials reduce the chances of rupture of the sheet.

The stent can also be arranged to reduce slippage as compared to a known covered stent. Firstly, the folds may provide an uneven outer surface which reduces slippage. Secondly, the outer surface may be provided with a high degree of friction, for example by selection of the biocompatible material of the stent or by roughening the outer surface.

The stent is particularly useful for use in the oesophagus, where rapid tissue in-growth is a particular problem, or as a stent graft in the aorta, for example to treat an Abdominal Aortic Aneurism. However, the stent may be used at any site in the body by appropriate design of the stent. The design of the stent is generic, so it can be adapted for use at different anatomical sites. For example, by varying the diameter, length and/or bifurcation the stent may be collapsed for retrieval at a later date after implantation.

Many different variations on the pattern of folds are possible. The choice of pattern may be selected to balance the ease of deployment, which generally improves as the degree of overlap in the folded pattern decreases, with the compactness of the stent when collapsed, which generally improves as the degree of overlap in the folded structure increases.

### BRIEF DESCRIPTION OF THE DRAWINGS

In the drawings:

FIG. 1 is a perspective view of a stent comprising a sheet folded with one of the folding patterns in accordance with the present invention;

FIG. 2 is a diagram of a unit cell of a pattern of folds with the sheet in its unfolded state when it is developed;

FIG. 3 is a diagram of the sheet with the unit cell of FIG. 2 developed to form the overall pattern of folds with the sheet in its unfolded state;

FIG. 4 is a progression of end views of the stent of FIG. 1 during expansion and contraction;

FIGS. 5, 7, 12, 13, 15-19, 21-24, 26-31, 33 and 34 are diagrams of unit cells with alternative patterns of folds with the sheet in its unfolded state;

FIGS. 6, 8, 14, 20, 25, 32, and 35-42 are diagrams of sheets with alternative patterns of folds with the sheet in its unfolded state;

FIGS. 9 to 11 are graphs of the change in dimensions of a stent against the number of unit cells in the pattern of folds;

FIG. 43 is a view of a portion of a sheet of the stent showing an aperture at a node where folds intersect;

FIG. 44 is a diagram of the sheet with the pattern of folds of FIG. 6 with a first type of frame;

FIG. 45 is a diagram of the sheet with the pattern of folds of FIG. 6 with a second type of frame;

FIGS. 46 and 47 are perspective views of a portion of stents with two different forms of frame;

FIG. 48 is a cross-section view of a portion of a stent with a further form of frame;

FIGS. 49, 51, 53 and 56 are diagrams of unit cells with further alternative patterns of folds with the sheet in its unfolded state;

FIGS. 50, 52, 54 and 55 are diagrams of sheets with the further alternative patterns of folds with the sheet in its unfolded state;

FIG. 57 is a diagram of a sheet with the Miura-Ori pattern of folds with the sheet in its unfolded state; and

FIG. 58 is a diagram illustrating how the Miura-Ori pattern of folds can be derived conceptually.

### DETAILED DESCRIPTION OF THE DRAWINGS

In order that the present invention may be better understood, the following description of embodiments of the present invention is given by way of non-limitative example with reference to the accompanying drawings.

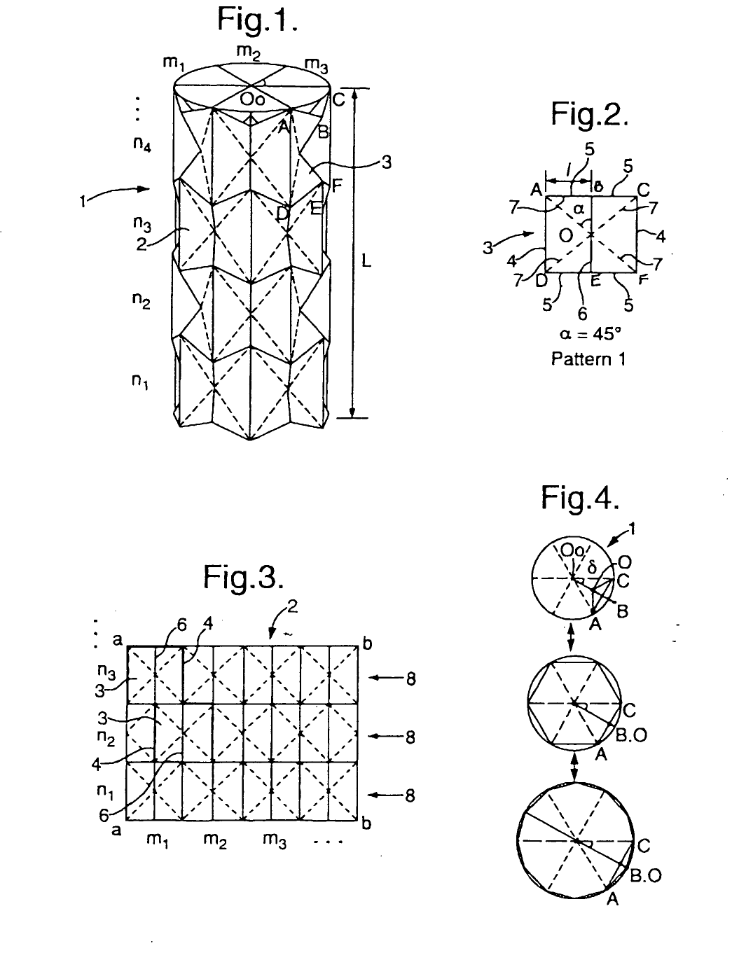

A stent 1 is illustrated in FIG. 1. The stent 1 comprises a biocompatible sheet 2. The sheet 2 has a tubular shape and is folded with a pattern of folds which allow the stent to be collapsed for deployment. The stent 1 may optionally further comprises a frame 12 which reinforces the sheet 2 and is described below, but first the sheet 2 will be described.

The pattern of folds of the sheet 2 comprises a unit cell 3 which is repeated over the entire area of the sheet 2. The pattern of folds is illustrated more clearly in FIGS. 2 and 3 which are views of, respectively, the unit cell 3 and the unit cells developed over the sheet 2 in the unfolded state, notionally “unwrapped” from its tubular form, the lines a-a and b-b being the same line longitudinally along the tubular shape of the sheet 2. The unit cells 3 are in rows repeating around a direction perpendicular to the longitudinal axis of the tubular shape of the sheet 2.

In FIGS. 2 and 3, and indeed the further figures illustrating patterns of folds, the lines are fold lines where the sheet 2 is folded. Between the folds, the sheet 2 is flat or planar. Continuous and dashed lines indicate folds of first and second opposite types. The two types are valley and hill folds. Hill folds are folds which form a peak when viewed from the outer side of the tubular shape of the sheet 2. Valley folds are folds which form a valley when viewed from the outer side of the tubular shape of the sheet 2. In the following description, it will be assumed that the folds of the first type are hill folds and the folds of the second type are valley folds.

In general, the two types of fold are reversible in any given pattern, that is replacing all hill folds with valley folds and replacing all valley folds with hill folds. However, some patterns when reversed cause the tubular shape of the sheet 2 to lock and hence do not allow the sheet 2 to be collapsed or expanded. The present invention contemplates the alternative that the folds of the first type are valley folds and the folds of the second type are hill folds, except when this causes locking of the structure.

For convenience, the pattern of folds illustrated in FIGS. 1 to 3 is referred to as Pattern 1.

The unit cell 3 comprises the following folds.

Unit cell 3 has an outer circumferential edge of hill folds. In particular, these are a pair of longitudinal edge folds 4 extending along the tubular shape of the sheet 2 parallel to one another and transverse edge folds 5 extending around the tubular shape of the sheet 2.

The unit cell 3 further comprises a central longitudinal fold 6 extending along the tubular shape of the sheet 2 between the transverse edge folds 5.

Lastly, the unit cell 3 has four angular folds 7 each extending from a respective intersection A, C, D or F of a longitudinal edge fold 4 with a transverse edge fold 5 to the central longitudinal fold 6. All four angular edge folds 7 intersect the central longitudinal fold 6 at the same position O. The length l of each transverse edge fold 5, that is from the intersection (e.g. at A) with a longitudinal edge fold 4 to a central intersection (e.g at B) with the central longitudinal fold 6, is equal to the length of the portion of the central longitudinal fold 6 from the central intersection (e.g. at B) with the transverse edge fold 5 to the intersection (e.g. at O) with an angular fold 7. Therefore, the triangle AOB and equivalent triangles within the unit cells 3 are isosceles triangles. The angle α (e.g. angle OAB) between a transverse edge fold 5 and an angular fold 7 is 45°.

The unit cell 3 is symmetrical about the central longitudinal fold 6 and about an imaginary line extending around the tubular shape of the sheet 2 perpendicular to the central longitudinal fold 6 and intersecting the central longitudinal fold 6 at O.

The angular folds 7 are valley folds and the central longitudinal fold 6 is a hill fold. Accordingly, the unit cell 3 is folded as illustrated in perspective view in FIG. 1 where the intersections A to F of the various fold lines from FIG. 2 are indicated for one of the unit cells 3.

The unit cell 3 is repeated as illustrated in FIG. 3. In particular, the unit cells 3 are arranged in rows 8 labelled n₁, n₂, . . . , the rows repeating along the tubular shape of the sheet 2. The unit cells 3 of adjacent rows are offset, as illustrated by the unit cells 3 illustrated in bold outline in FIG. 3, that is with the longitudinal edge folds 4 of each row 8 meeting the central longitudinal folds 6 of the adjacent rows 8. The number n of rows 8 labelled n₁, n₂, . . . in FIGS. 1 and 3 and the number m of unit cells 3 within each row around the tubular shape of the sheet 2 labelled m₁, m₂, . . . in FIGS. 1 and 3 can be freely varied. Similarly, the absolute dimensions of the sheet 2 and the unit cell 3 can be freely varied.

One of the interesting properties of Pattern 1 is that it causes the sheet 2 to collapse and expand both longitudinally and radially. That is both the length of the tubular shape of the sheet 2 and the radius of the tubular shape of the sheet 2 increase during expansion and decrease during collapse. This property provides the advantage that the folded stent 1 can be packaged compactly. This makes the stent 1 easier to deliver through narrow passages of the body and facilitates deployment at a blocked site where it can be expanded.

FIG. 4 is a progression of end views of the stent 1 during its expansion and contraction. As can be seen from FIG. 4, the central part of the unit cell 3 at the intersection (at O) of the angular fold 7 with the central longitudinal fold 6 moves inwardly and outwardly, causing a change in the radius of the sheet 2 during deployment. This also causes a reduction in the distance between the intersections (at B and E) between the central longitudinal fold 6 and the transverse edge folds 5, which causes a change in the axial length L of the sheet 2.

Further possible patterns of folds will now be described. The further patterns of folds are variations on Pattern 1 shown in FIGS. 1 to 3. For clarity and for brevity, the further patterns will all be described by explaining the variations from Pattern 1 without repeating the common features. The same reference numerals as for Pattern 1 will be used to denote the sheet 2, the unit cell 3, the equivalent folds 4 to 7 and the rows 8.

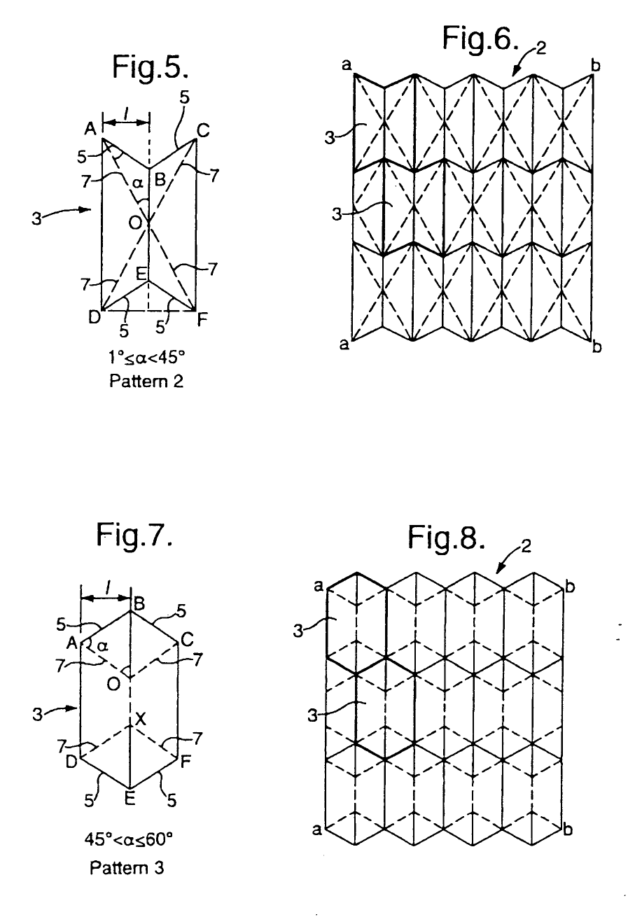

Pattern 2 is illustrated in FIGS. 5 and 6. FIG. 5 is a diagram of the unit cell 3 and FIG. 6 is a diagram of the sheet with the unit cell 3 developed across the sheet 2. Pattern 2 is similar to Pattern 1 except that the angle α (e.g. angle OAB) between each transverse edge fold 5 and angular fold 7 is less than 45°, so the unit cell 3 is no longer rectangular.

Pattern 3 is illustrated in FIGS. 7 and 8. FIG. 7 is a diagram of the unit cell 3 and FIG. 8 is a diagram of the sheet 2 with the unit cell 3 developed across the sheet 2.

Pattern 3 varies from Pattern 1 in that the angle α (e.g. angle OAB) between each transverse edge fold 5 and in respect of angular fold 7 is greater than 45° and less than or equal to 60°. As a result the shape of the unit shape 3 becomes a polygon. The angle α should be equal to or less than 60° to allow folding of the sheet 2.

Pattern 3 also varies from Pattern 1 in that the angular folds 7 do not all intersect the central longitudinal fold 6 at the same position. Instead, for each pair of angular folds 7 at opposite longitudinal ends of the unit cell 3, the pair of angular folds 7 intersect the central longitudinal folds 6 at the same position, but the pairs of angular folds 7 intersect the central longitudinal fold 6 at separated positions O and X. Between these separated positions O and X, the central longitudinal fold 6 is a valley fold. However, the portions of the central longitudinal fold 6 extending from a central intersection (at B or E) with a respective one of the transverse edge folds 5 to a respective intersection (at O or X) with the angular folds 7 remain as hill folds. The separation between the intersections (at O and X) of each pair of angular folds 7 and the central longitudinal fold 6 may be freely varied. This separation may be reduced to zero (as in Patterns 1 and 2), but the longitudinal length of the unit cell 3, or more particularly the length of the central longitudinal fold 6, may not be further reduced or else folding is prevented.

To understand and compare the folding of Patterns 1 to 3, the geometric properties of Patterns 1 to 3 have been analysed as follows. The analysis is based on Pattern 2 with the angle α as 30° and on Pattern 3 with the angle α as 60°.

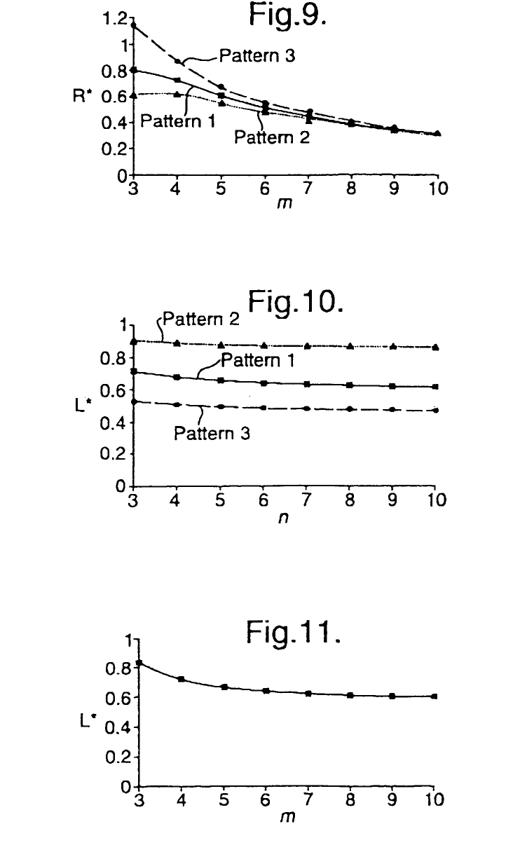

Firstly, the ratio R* of the outer radius of sheet 2 (ie the distance from Oo to A or B) in its fully folded configuration to the outer radius of the sheet 2 in its fully deployed configuration was calculated for stents 1 having differing numbers m of unit cells 3 in each row 8 of the sheet 2 around the tubular shape of the sheet 2. The relationship between R* and m for Patterns 1, 2 and 3 is illustrated in FIG. 9 where Pattern 1 is shown by a continuous line, Pattern 2 is shown by a dotted line and Pattern 3 is shown by a dashed line.

For each pattern, it will be noted that the value of R* decreases as the number m of unit cells 3 in each row 8 increases. In other words, a large value of m makes the pattern fold more compact in the radial direction. Thus the number m of unit cells 3 in each row 8 around the tubular shape of the sheet 2 is preferably large to minimise the radius of the sheet 2 on collapse. However, increasing the number m of unit cells 3 in each row 8 causes the folding to become complex and potentially affected by the thickness of the material of the sheet 2. The number m of unit cells 3 in each row 8 should be selected to balance these two factors.

It will also be noted from FIG. 9 that as compared to Pattern 1, Pattern 2 has a lower value of R* and hence folds more compactly, whereas Pattern 3 has a higher value of R* and hence folds less compactly. However, the difference in the value of R* between Patterns 1, 2 and 3 becomes small when m is larger than 9. When m=10 the radius of the sheet 2 in its fully folded configuration is about 30% of that in its fully deployed configuration, for each pattern.

Also, the value L* of the ratio of the total length of the sheet 2 in its fully folded configuration to the length of the sheet 2 in its fully deployed configuration was calculated for different values of the number m of unit cells in each row 8 of the sheet 2 around the tubular shape of the sheet 2 and for differing values of the number n of rows 8 along the tubular shape of the sheet 2.

FIG. 10 shows the value of L* for each of Patterns 1 to 3 for differing values of n when m=6. In FIG. 10, Pattern 1 is shown by a continuous line, the Pattern 2 is shown by a dotted line and Pattern 3 is shown by a dashed line. It will be seen that for each pattern, the ratio L* slowly decreases as n increases. This means that all three Patterns fold more compactly in the longitudinal direction as the number n of rows 8 of unit cells 3 increases. The value of L* becomes nearly constant when n is greater than 7, so there is no particular benefit in increasing the number n of unit cells 3 above about 7.

It will be noted that, as compared to Pattern 1 in the longitudinal direction, Pattern 3 folds more compactly, whereas Pattern 2 folds less compactly but maintains flexibility. Therefore, pattern 3 is preferred for uses where longitudinal collapse is desirable to allow access of the stent 1 to the blocked site, whereas Pattern 2 is preferred for uses where the medical practitioner prefers the longitudinal collapse to be minimised.

FIG. 11 shows the value of L* for Pattern 1 for different values of m when n=7. It will be noted that L* becomes smaller as m increases. Thus increasing m reduces the longitudinal collapse of the sheet 2 when folded, as well as reducing the radial collapse.

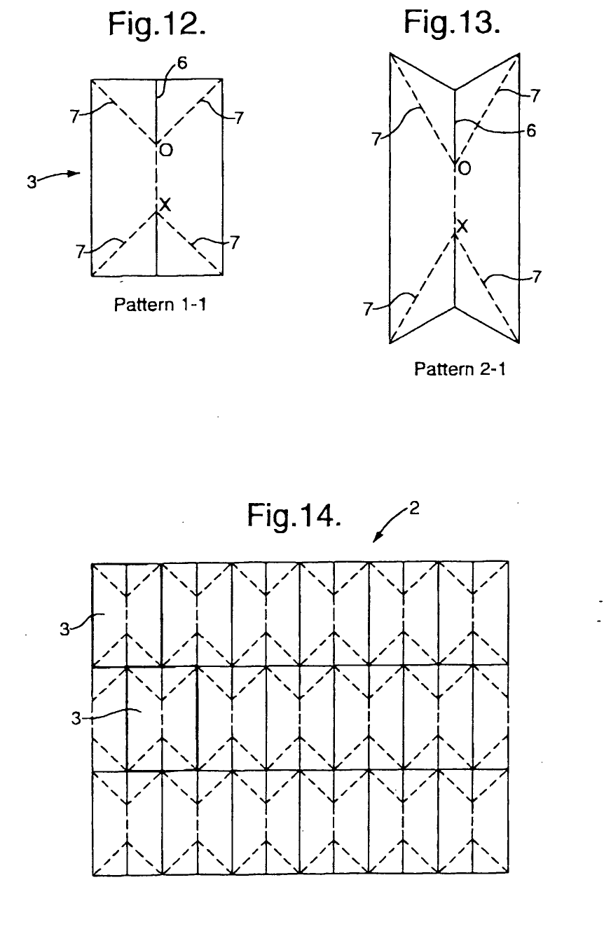

FIGS. 12 and 13 are diagrams of the unit cells 3 of the Patterns 1-1 and 2-1 which are variations of Patterns 1 and 2, respectively. FIG. 14 is a diagram of Pattern 1-1 developed across the sheet 2. In both cases, the length of the unit cell 3 is increased so that the pairs of angular folds 7 intersect the central longitudinal fold 6 at separated positions O and X between which the central longitudinal fold 6 is a valley fold.

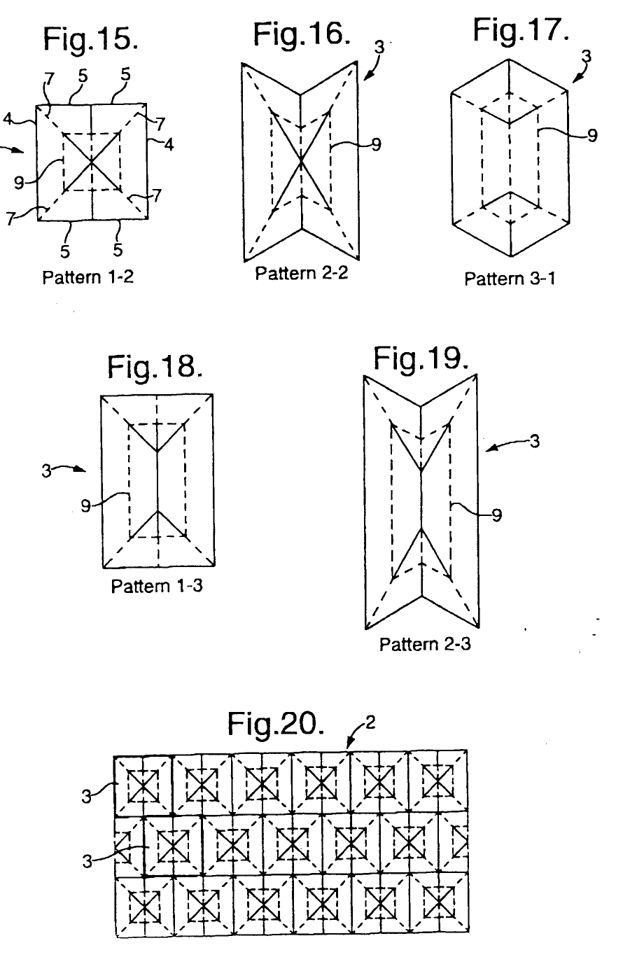

FIGS. 15 to 19 are diagrams of the unit cell 3 of Patterns 1-2, 2-2, 3-1, 1-3 and 2-3, respectively, which are themselves variations of Patterns 1, 2, 3, 1-1 and 2-1, respectively.

FIG. 20 is a diagram of Pattern 1-2 with the unit cell 3 developed across the sheet of material 2. In each case, the variation is to provide an additional ring of valley folds 9. Each valley fold 9 extends parallel to an adjacent longitudinal or transverse edge fold 4 or 5. The valley folds 9 extends between an angular fold 7 and either another angular fold 7 or else the central longitudinal fold 6. The ring of valley folds 9 causes the surface of the unit cell 3 to be folded twice. Therefore inside the ring of valley folds 9, the folds of the basic unit cell 3, that is the angular fold 7 and the central longitudinal fold 6, reverse. That is to say, hill folds reverse to valley folds and valley folds reverse to hill folds. Such a ring of valley folds 9 has the advantages that the double folding pattern causes the inner surface of the sheet 2 inside the tubular shape of the sheet 2 to become smoother and allows the unit cell 3 to be folded more compactly, because the peak point O of the unit cell 3 in its folded state shown in FIG. 4 is folded inside points A and C of the folded unit cell 3, ie allowing the unit cells 3 to be folded compactly in the radial direction.

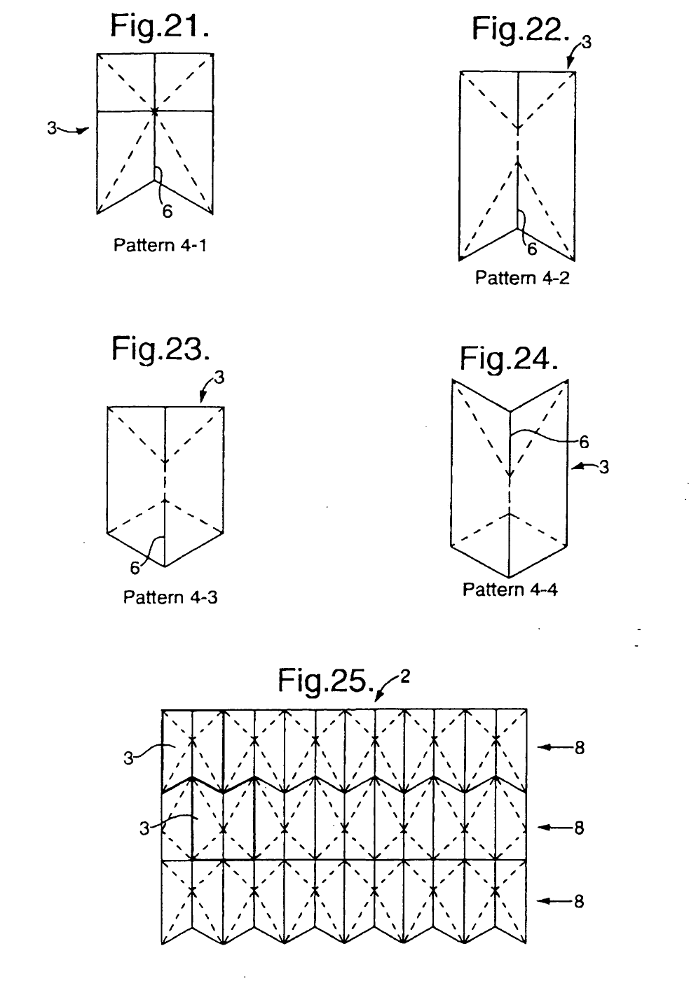

The unit cells 3 described above are symmetrical both about the central longitudinal fold 6 and also about an imaginary line extending around the tubular shape of the sheet 2 perpendicular to the central longitudinal fold 6. However, this is not essential. Either or both degrees of symmetry may be removed. For example FIGS. 21 to 24 are diagrams of Patterns 4-1 to 4-4, respectively, which are symmetrical only about the central longitudinal fold 6. FIG. 25 is a diagram of Pattern 4-1 with the unit cell 3 developed over the sheet 2. Accordingly, the unit cell 3 of alternate rows 8 is reversed in the longitudinal direction. This may also be viewed as a Pattern having a larger unit cell comprising the two unit cells 3 illustrated in FIG. 21 in bold outline combined together. Patterns 4-1 to 4-4 may also be viewed as consisting of the other half of one of the Patterns described above with the lower of another of the Patterns described above. For example, Pattern 4-1 may be viewed as the upper half of Pattern 1 combined with the lower half of Pattern 2, and so on.

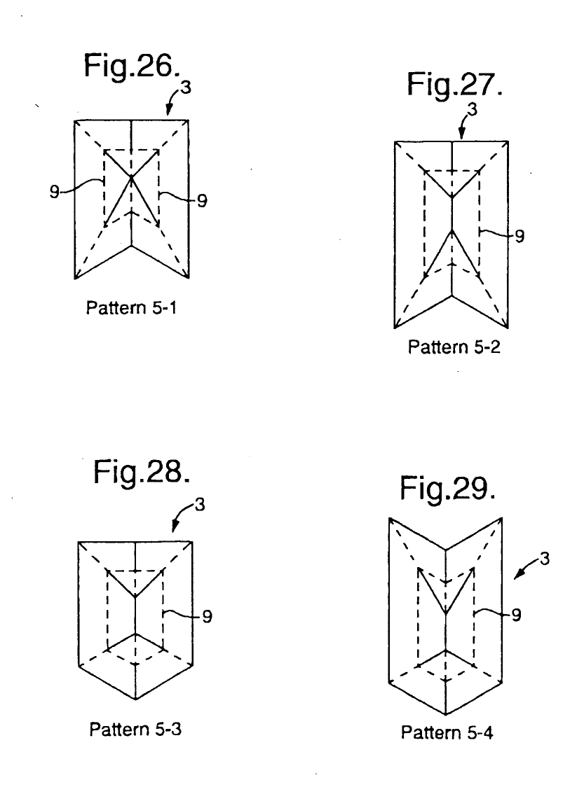

FIGS. 26 to 29 are diagrams of the unit cell 3 of Patterns 5-1 to 5-4, respectively, which are variations of Patterns 4-1 to 4-4, respectively, the variation is that the unit cell 3 further comprises a ring of valley folds 9 as in Patterns 1-2, 2-2, 3-1, 1-3 and 2-3.

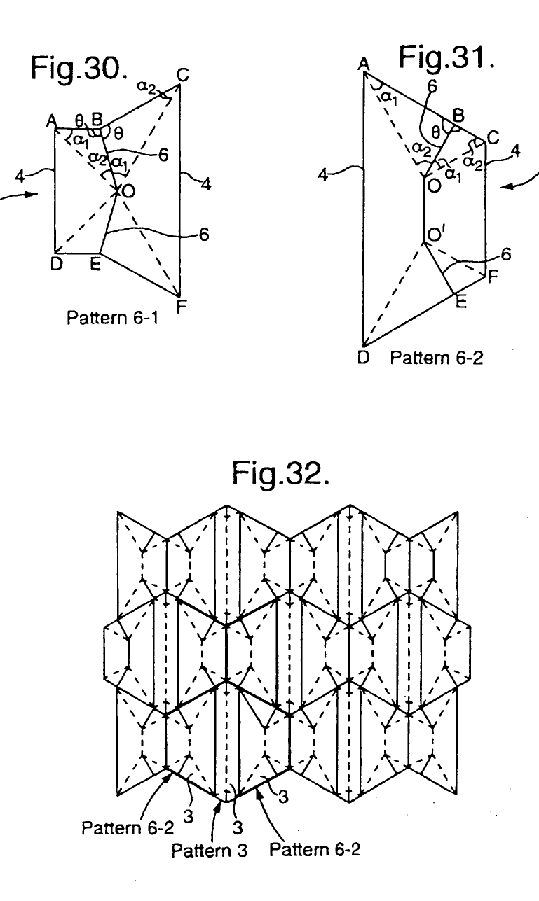

FIGS. 30 and 31 illustrate Patterns 6-1 and 6-2 which are symmetrical only about an imaginary line extending around the tubular shape of the sheet 2. These Patterns may also be viewed as combinations of longitudinally-extending halves of different Patterns described above, except that the central longitudinal fold 6 extends at an angle to the longitudinal direction along which the longitudinal edge folds 4 extend. In particular, if the angle BAO is α₁, and then the angle BCO is α₂, then the angle AOB is α₂, the angle BOC is α₁, and both angles ABO and CBO are (π−α₁−α₂). For example, Pattern 6-1 may be viewed as the combination of the left half of Pattern 1 with the right half of Pattern 2. Similarly, Pattern 6-2 may be viewed as the combination of the left half of Pattern 2-1 and the right half of Pattern 3.

Unlike the previous patterns. Pattern 6-2 cannot be used by itself, but must be combined with another pattern. For example, FIG. 32 is a diagram of Pattern 6-2 with the unit cell 3 developed over a sheet 2 and combined with Pattern 3. To enable the unit cells to fit together, alternate unit cells 3 of Pattern 6-2 along each row 8 are longitudinally reversed and a unit cell of Pattern 3 is arranged between successive pairs of unit cells 3 of Pattern 6-2, between the longer longitudinal edges of the unit cells 3 of Pattern 6-2. Thus, a larger unit cell is formed by the combination of two unit cells 3 of Pattern 6-2 with a unit cell of Pattern 3.

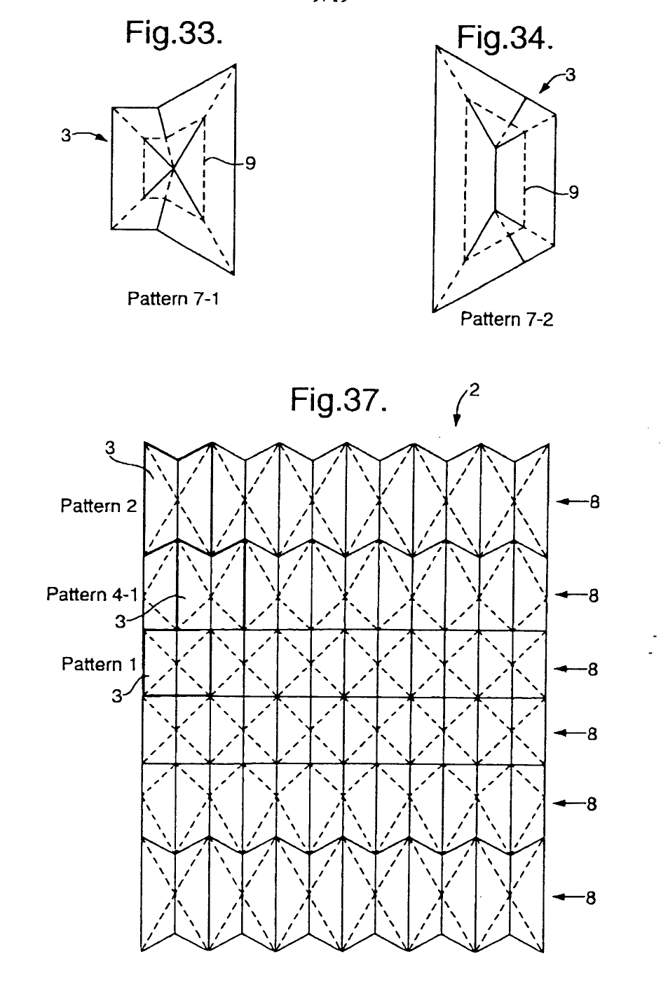

FIGS. 33 and 34 are diagrams of the unit cell 3 of Patterns 7-1 and 7-2 which are variations of Patterns 6-1 and 6-2. The variation is the addition of a ring of valley folds 9 similar to the valley folds 9 of Patterns 1-2, 2-2, 3-1, 1-3 and 2-3.

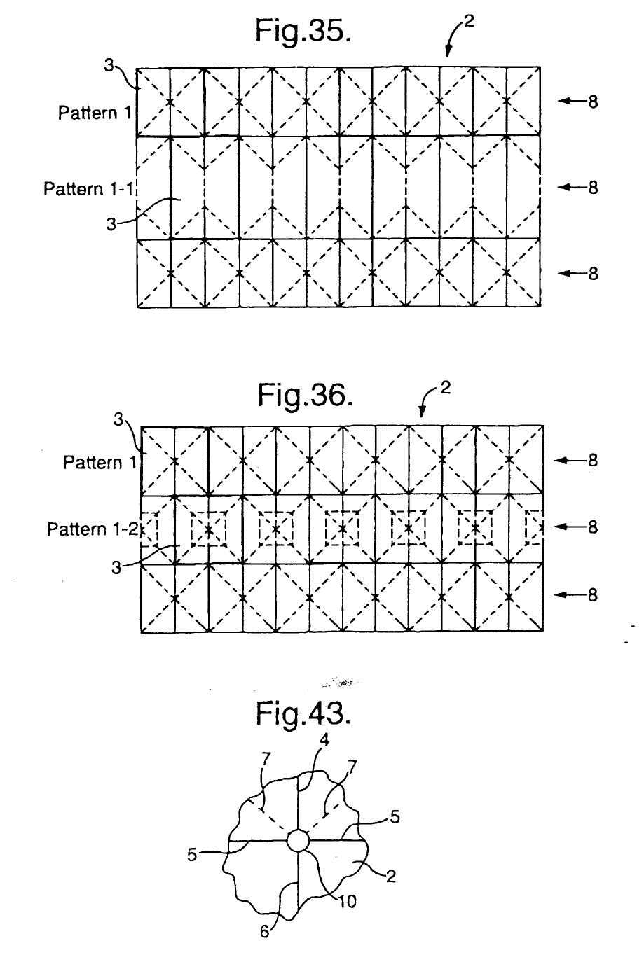

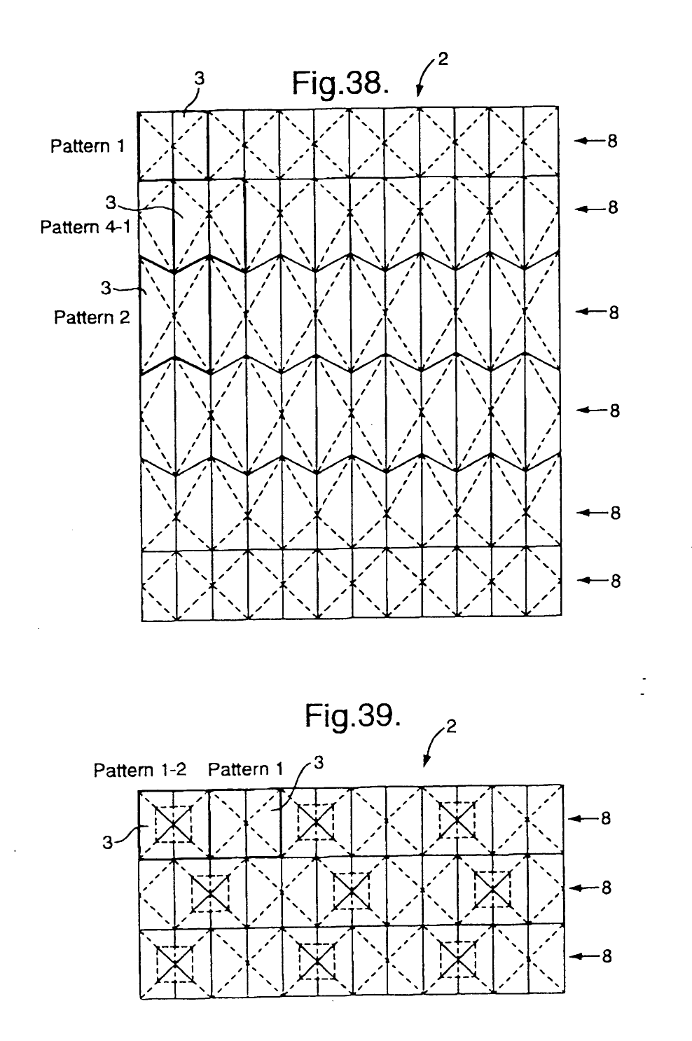

In the Patterns described above, a single unit cell 3 is repeated over the entire sheet, but this is not essential. In fact, different unit cells 3 may be repeated over different portions of the sheet 2. For example, FIGS. 35 to 39 show patterns of folds in which different rows 8 comprise a respective, different unit cell 3 repeated around the tubular shape of the sheet 2. In FIGS. 35 and 36, two different patterns are used. In FIG. 35, Patterns 1 and 1-1 are used for alternate rows. In FIG. 36, Patterns 1 and 1-2 are used for alternate rows. In FIGS. 37 and 38, three different patterns are used. In particular, in both FIGS. 37 and 38 unit cells 3 of Patterns 1, 4-1 and 2 are used for different respective rows 8, although in a different order longitudinally along the tubular shape of the sheet 2.

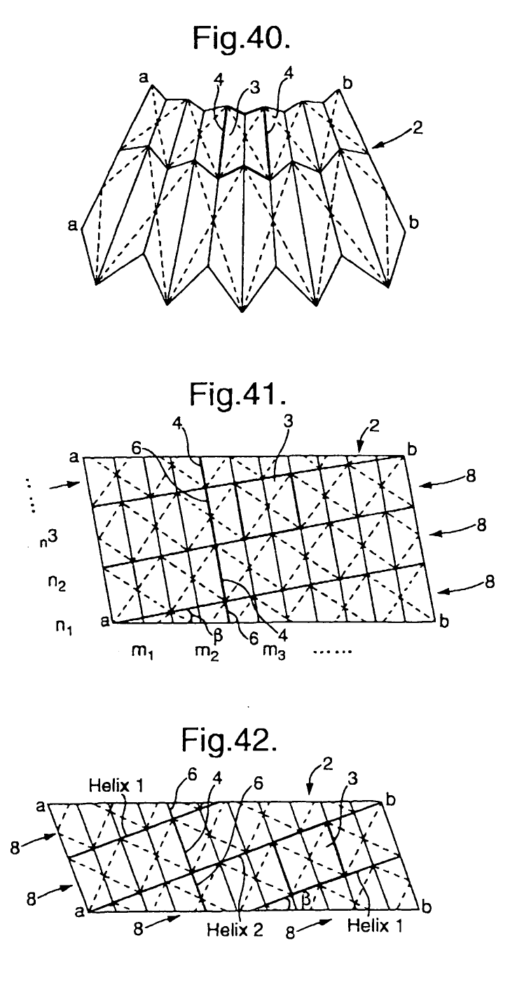

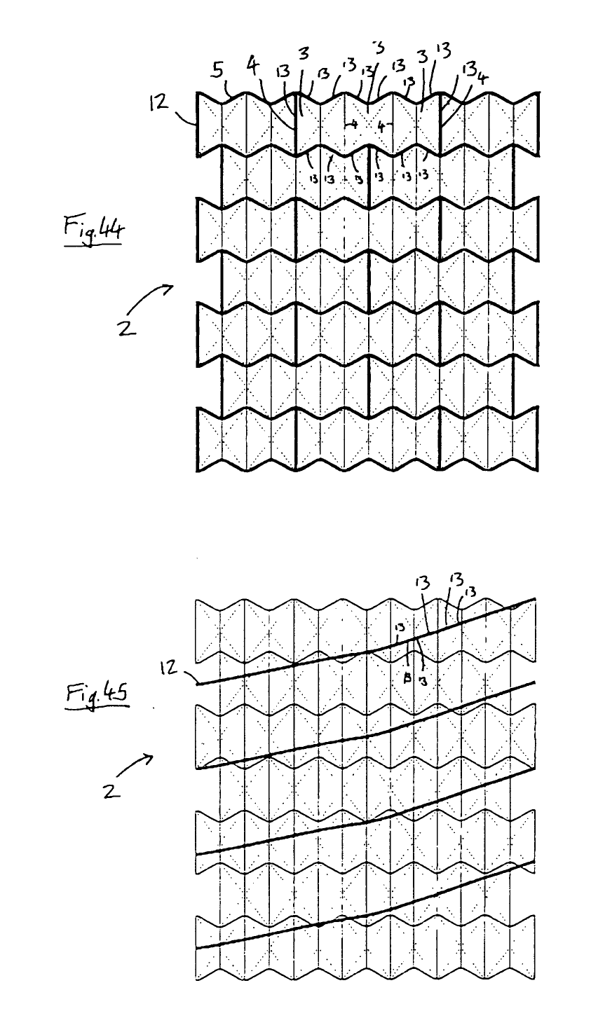

Similarly, FIG. 39 is a diagram of a pattern of folds in which each row 8 comprises two different unit cells 3 alternating along the row 8, in particular the unit cells of Patterns 1 and 1-2.

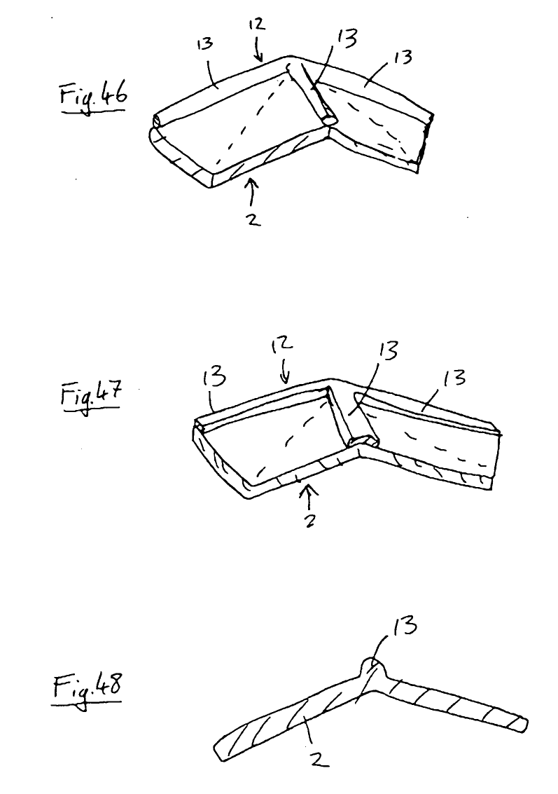

The patterns of folds described above provide the sheet 2 with a tubular shape which is generally cylindrical by means of the unit cells 3 being arranged with parallel longitudinal edge folds 4 and has the same radius along the length of the tubular shape of the sheet 2. However, this is not essential. For example, the sheet 2 may be arranged with a tubular shape which is conical along the entire length or along a portion thereof. This may be achieved using the pattern of folds illustrated in FIG. 40 which is based on a unit cell 3 of Pattern 2, but in which the unit cells 3 are of different sizes with the longitudinal edge folds 4 being angled relative to one another, instead of parallel. Therefore the longitudinal edge folds 4 are also angled with respect to the longitudinal direction of the tubular shape of the sheet 2. As a result, the sheet 2 of FIG. 40 forms a conical (or frustoconical) tubular shape when folded. Alternatively, the sheet 2 may have a more complicated structure, for example having plural tubular portions branching off from a common node.

FIGS. 41 and 42 are diagrams of patterns in which unit cells 3 are arranged on the sheet 2 in rows 8 which progress helically around the tubular shape of the sheet 2 when the sheet 2 is folded. FIGS. 41 and 42 are based on a unit cell of Pattern 1, but any of the patterns described above could alternatively be used. Consequently, the rows 8 of unit cells are arranged at a pitch angle or helix angle β which is the angle between the direction in which the unit cells repeat and plane perpendicular to the longitudinal axis of the tubular shape of the sheet 2. When the sheet 2 is folded with the opposite lines a-a and b-b in FIGS. 41 and 42 being the same line, successive rows 8 of unit cells 3 join end-to-end to form a longer row which progresses helically around the tubular shape of the sheet 2. In the pattern of FIG. 41, the angle x is selected so that the rows 8 combine to form a single row progressing helically around the tubular shape of the sheet 2. In the pattern of FIG. 42, the angle β is selected so that the rows 8 join together to form two rows progressing helically around the tubular shape of the sheet 2.

As a result of the helical pattern it will also be noted that the longitudinal edge folds 4 and the central longitudinal folds 6 of alternate rows 8 meet together to form an uninterrupted fold line which also progresses helically around the tubular shape of the sheet 2.

Such a helical structure provides a number of advantages. Firstly, it allows the sheet 2 to be folded compactly in the longitudinal direction because of its capability of torsion. Secondly, the helical pattern assists with deployment, because the expansion and collapse of the sheet 2 is usually synchronised over the area of the sheet 2. That is to say, the helical progression of the pattern of folds spreads the force causing expansion or collapse to be transmitted along the length of the tubular shape of the sheet 2. This may be viewed as the force being transmitted along the uninterrupted lines of folds formed by the longitudinal edge folds 4 and the central longitudinal folds 6 of alternate rows 8 which progress helically around the tubular shape of the sheet 2. This means that a twist applied to the sheet 2 can be used to generate expansion or collapse of the sheet 2 which greatly assists deployment of the stent 1 because a twist is simple to perform. Thirdly, the helical structure holds the sheet 2 in its expanded configuration. This is because collapse of the stent requires torsional forces which are not usually developed at sites in the body.

The patterns described above are preferred because of their simplicity and hence ease of design and manufacture. However a stent in accordance with the present invention may be formed using numerous other patterns of folds which allow radial collapse and optionally longitudinal collapse. Alternative patterns may be regular or irregular and the sheet between the folds may in general be flat or curved.

Some examples of further patterns which are based on a modification of the patterns described above will now be described.

Pattern 8 is illustrated in FIGS. 49 and 50. FIG. 49 is a diagram of the unit cell 3 and FIG. 50 is a diagram of the sheet 2 with the unit cell 3 developed across the sheet 2 in the unfolded state, notionally “unwrapped” from its tubular form, the lines a-a and b-b being the same line longitudinally along the tubular shape of the sheet 2.

In Pattern 8, the unit cell 3 comprises the following folds.

The unit cell 3 has a first longitudinal fold 20 which is a hill fold and a second longitudinal fold 21 which is a valley fold, the first and second longitudinal folds 20 and 21 extending away from a common point O. The longitudinal folds 20 and 21 extend along the tubular shape of the sheet 2. In this example, the longitudinal folds 20 and 21 are collinear and so form a straight uninterrupted fold line, but this is not essential.

The unit cell 3 also has an outer circumferential ring of four edge folds 22 and 23 which are hill folds and consist of two major edge folds 22 and two minor edge folds 23. The two minor edge folds 23 intersect at point E at the outer end of the first longitudinal fold 21, although in this example the two minor edge folds 23 are collinear and so form a straight uninterrupted fold line. The two major edge folds 22 intersect at point B at the outer end of the second longitudinal fold 20. One major edge fold 22 and one minor edge fold 23 are arranged on each side of the longitudinal folds 20 and 21, intersecting at points D and F, respectively.

The terms “major” and “minor” are used merely to distinguish between the major edge folds 22 and the minor edge folds 23. The major edge folds 22 are generally longer than the minor edge folds 23, but this is not always the case in all variations of Pattern 8.

Lastly, the unit cell 3 has two angular folds 24 which are valley folds each extending from a respective intersection D or F of a major edge fold 22 with a minor edge fold 23 to the common point O from which the first and second longitudinal folds 20 and 21 extend. Thus, the first and second longitudinal folds 20 and 21 and the angular folds 24 all intersect at the common point O.

In this example the unit cell 3 is symmetrical about the central longitudinal folds 20 and 21 but this is not essential.

Thus, Pattern 8 may be considered as a modification of Pattern 1 in which points A and C of Pattern 1 are drawn inwards to coincide with point B of Pattern 1 so that the transverse edge folds 4 of Pattern 1 formed between points A and B at one end of the unit cell 3 disappear and so that the angular folds 7 of Pattern 1 formed between points A and O and between points B and O at one end of the unit cell 3 overlie the longitudinal fold 6 of Pattern 1. When considered in this manner, the major edge folds 22 of Pattern 8 correspond to the longitudinal edge folds 4 of Pattern 1; the minor edge folds 23 of Pattern 8 correspond to the transverse edge folds S of Pattern 1; the longitudinal folds 20 and 21 of Pattern 8 correspond to the central longitudinal fold 4 of Pattern 1, and the angular folds 24 of Pattern 8 correspond to the angular folds 7 of Pattern 1.

Other than this modification, Pattern 8 is the same as Pattern 1 and the above description of Pattern 1 and the variations to Pattern 1 apply equally to Pattern 8. Thus, in Pattern 8, the pattern of folds of the sheet 2 comprises a unit cell 3 which is repeated over the entire area of the sheet 2. In particular, the unit cells 3 are in a plurality of rows 8 repeating around a direction perpendicular to the longitudinal axis of the tubular shape of the sheet 2. The unit cells 3 of adjacent rows 8 are aligned, that is with the first longitudinal fold 20 of any given unit cell 3 meeting the second longitudinal fold 21 of a unit cell 3 in an adjacent row 8. In this arrangement, the minor edge folds 23 may be thought of as extending around the tubular shape of the sheet 2, and the major edge folds 22 may be thought of as extending along the tubular shape of the sheet 2 albeit at an acute angle to the longitudinal axis.

As for Pattern 1, in Pattern 8 and the following figures illustrating variations to Pattern 8, the lines are fold lines where the sheet 2 is folded. Between the folds, the sheet 2 is flat or planar. Continuous and dashed lines indicate folds of first and second opposite types. The two types are valley and hill folds. Hill folds are folds which form a peak when viewed from the outer side of the tubular shape of the sheet 2. Valley folds are folds which form a valley when viewed from the outer side of the tubular shape of the sheet 2. In the following description, it will be assumed that the folds of the first type are hill folds and the folds of the second type are valley folds.

In general, the two types of fold are reversible in any given pattern, that is replacing all hill folds with valley folds and replacing all valley folds with hill folds. However, some patterns when reversed cause the tubular shape of the sheet 2 to lock and hence do not allow the sheet 2 to be collapsed or expanded. The present invention contemplates the alternative that the folds of the first type are valley folds and the folds of the second type are hill folds, except when this causes locking of the structure.

As before, the number of rows 8 and the number of unit cells 3 within each row 8 around the tubular shape of the sheet 2 can be freely varied. Similarly, the absolute dimensions of the sheet 2 and the absolute and relative dimensions of the unit cell 3 can be freely varied.

Pattern 8 causes the sheet 2 to collapse and expand both longitudinally and radially. That is both the length of the tubular shape of the sheet 2 and the radius of the tubular shape of the sheet 2 increase during expansion and decrease during collapse. This property provides the advantage that the folded stent 1 can be packaged compactly. This makes the stent 1 easier to deliver through narrow passages of the body and facilitates deployment at a blocked site where it can be expanded.

That being said, Pattern 8 does not fold as efficiently as Pattern 1 with the effect that the degree of collapse of the stent 1 is less with Pattern 8 than with Pattern 1 for a unit cell 3 of comparable length along the longitudinal axis of the tubular shape of the stent 1.

As previously noted, Pattern 8 can be varied in a similar manner to Pattern 1. Some further patterns of folds which are variations on Pattern 8 will now be described. For clarity and for brevity, the further patterns will all be described by explaining the variations from Pattern 8 without repeating the common features. The same reference numerals as for Pattern 8 will be used to denote the sheet 2, the unit cell 3, the equivalent folds 20 to 24 and the rows 8.

Pattern 9 is illustrated in FIGS. 51 and 52. FIG. 51 is a diagram of the unit cell 3 and FIG. 52 is a diagram of the sheet with the unit cell 3 developed across the sheet 2. Pattern 9 is similar to Pattern 8 except that the minor edge folds 23 are not collinear and the respective angles between the minor edge folds 23 and the first longitudinal fold 20 inside the unit cell 3 (angles OEF and OED) are obtuse.

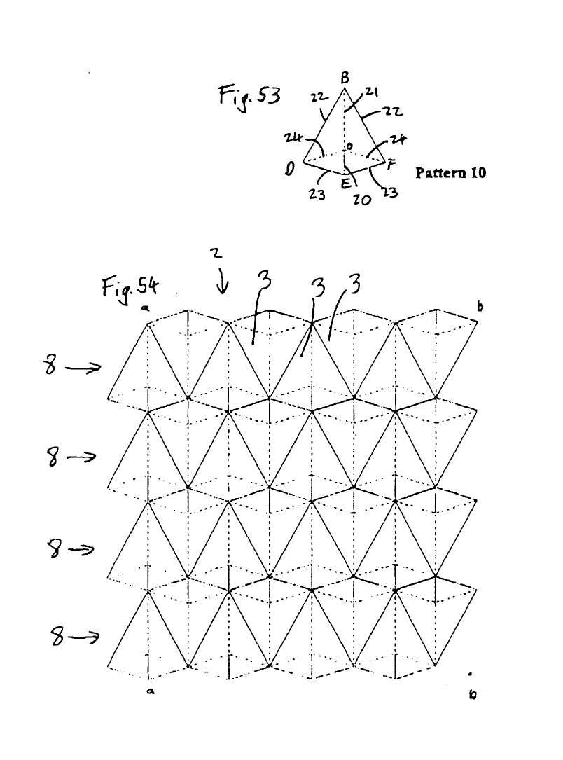

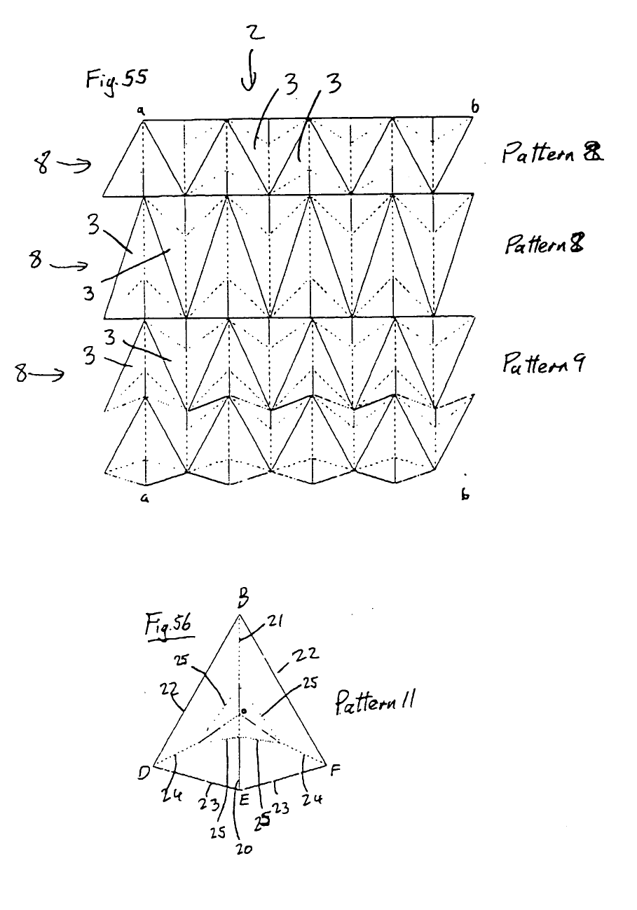

Pattern 10 is illustrated in FIGS. 53 and 54. FIG. 53 is a diagram of the unit cell 3 and FIG. 54 is a diagram of the sheet 2 with the unit cell 3 developed across the sheet 2. Pattern 10 is similar to Pattern 8 except that the minor edge folds 23 are not collinear and the respective angles between the minor edge folds 23 and the first longitudinal fold 20 inside the unit cell 3 (angles OEF and OED) are obtuse.

FIG. 56 is a diagram of the unit cell 3 of Pattern 11 which is a variation of Pattern 10. The variation is to provide an additional ring of valley folds 25. Each valley fold 25 extends inside an adjacent major edge fold 22 or minor edge fold 23, between an angular fold 24 and either the first or second longitudinal fold 20 or 21. The ring of valley folds 25 causes the surface of the unit cell 3 to be folded twice. Therefore inside the ring of valley folds 25, the folds of the basic unit cell 3, that is the angular fold 24 and the longitudinal folds 20 and 21, reverse. That is to say, hill folds reverse to valley folds and valley folds reverse to hill folds. Such a ring of valley folds 25 has the advantages that the double folding pattern causes the inner surface of the sheet 2 inside the tubular shape of the sheet 2 to become smoother and allows the unit cell 3 to be folded more compactly, because the peak point O of the unit cell 3 in its folded state is folded inside points D and F of the folded unit cell 3, ie allowing the unit cells 3 to be folded compactly in the radial direction.

Valley folds 25 of the same nature may equally be applied to Pattern 8 and any other variation of Pattern 8.

The unit cells 3 of Patterns 8 to 11 are symmetrical about the first and second longitudinal folds 20 and 21 which are themselves collinear. However, this is not essential. The symmetry may be removed so that the unit cell 3 has a different configuration on each side of the first and second longitudinal folds 20 and 21.

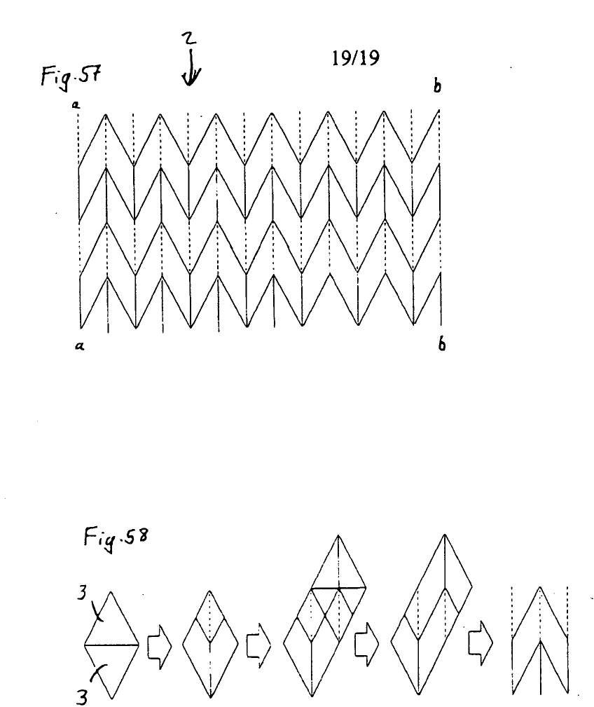

In the Patterns 8 to 11, an identical unit cell 3 is repeated over the entire sheet 2, but this is not essential. In fact, different unit cells 3 may be repeated over different portions of the sheet 2. For example, FIG. 55 shows a pattern of folds in which different rows 8 comprise a respective, different unit cell 3 repeated around the tubular shape of the sheet 2. The first two rows 8 are unit cells 3 of Pattern 8 with different length unit cells 3 and the third row 8 is unit cells 3 of Pattern 9. In a similar manner, it is possible to combine rows 8 of unit cells 3 in accordance with Patterns 1 to 7 (or variations thereof) with rows S of unit cells 3 in accordance with Patterns 8 to 11 (or variations thereof).

The patterns of folds described above provide the sheet 2 with a tubular shape which is generally cylindrical by means of the unit cells 3 being arranged with parallel longitudinal edge folds 4 and has the same radius along the length of the tubular shape of the sheet 2. However, this is not essential. For example, the sheet 2 may be arranged with a tubular shape which is conical along the entire length or along a portion thereof. This may be achieved using a pattern of folds in which the unit cells 3 are of different sizes and angled in a similar manner to the pattern shown in FIG. 40, so that the sheet 2 forms a conical (or frustoconical) tubular shape when folded. Such a shape has the advantage of improving anchoring of the stent 1 at some sites.

Alternatively, the sheet 2 may have a more complicated structure, for example having plural tubular portions branching off from a common node.

Another possible variation is that the unit cells 3 are arranged on the sheet 2 in one or more rows 8 which progress helically around the tubular shape of the sheet 2 when the sheet 2 is folded in a similar manner to FIGS. 41 and 42. As a result of the helical pattern it will also be noted that the minor edge folds 23 of adjacent each turn of the rows 8 meet together to form an uninterrupted fold line which also progresses helically around the tubular shape of the sheet 2.

Such a helical structure provides a number of advantages. Firstly, it allows the sheet 2 to be folded compactly in the longitudinal direction because of its capability of torsion. Secondly, the helical pattern assists with deployment, because the expansion and collapse of the sheet 2 is usually synchronised over the area of the sheet 2. That is to say, the helical progression of the pattern of folds spreads the force causing expansion or collapse to be transmitted along the length of the tubular shape of the sheet 2. This may be viewed as the force being transmitted along the uninterrupted lines of folds formed by the longitudinal edge folds 4 and the central longitudinal folds 6 of alternate rows 2 which progress helically around the tubular shape of the sheet 2. This means that a twist applied to the sheet 2 can be used to generate expansion or collapse of the sheet 2 which greatly assists deployment of the stent 1 because a twist is simple to perform. Thirdly, the helical structure holds the sheet 2 in its expanded configuration. This is because collapse of the stent requires torsional forces which are not usually developed at sites in the body.

One further pattern of folds which may be applied to the sheet 2 is shown in FIG. 57 which is a diagram of a sheet 2 in the unfolded state, notionally “unwrapped” from its tubular form, the lines a-a and b-b being the same line longitudinally along the tubular shape of the sheet 2. This pattern of folds is based on the Miura-Ori pattern of folds known for folding a planar (ie not tubular) sheet, for example a map. As before, continuous lines indicate hill folds and dotted lines indicate valley folds, although the entire pattern may be reversed. FIG. 58 shows how the Miura-Ori pattern of folds can be derived conceptually.

Optionally, the stent 1 fisher comprises a frame 12 which reinforces the sheet 2. Two types of frame 12 are illustrated in FIGS. 44 and 45 which are views of, the sheet 2 in the unfolded state, notionally “unwrapped” from its tubular form. FIGS. 44 and 45 illustrate the sheet 2 as being folded with Pattern 2 shown in FIG. 6 but this is merely an example and the frame 12 is also used to reinforce the sheet 2 when folded with any other folding pattern including all the Patterns described above.

In both types of frame 12 shown in FIGS. 44 and 45, the frame 12 comprises an arrangement of elongate members 13 which lie along the sheet 12 and fold relative to one another in conformity with the sheet 2. In this example, the frame 12 extends continuously between the elongate members 13 so the division of the frame 12 into elongate members 13 is notional. The boundaries between the elongate members 13 occur in every location where the frame 12 crosses one of the folds 4 to 7. As the elongate members 13 fold with the sheet 2, this allows the frame 12 to be collapsed together with the sheet 2 for deployment of the stent 1. The frame 12 extends around the tubular shape of the sheet 2 in the folded state and therefore reinforces the sheet 2.

In the first type of frame 12 shown in FIG. 44, the elongate members 13 extend along longitudinal edge folds 4 and transverse edge folds 7 which form part of the outer circumferential edges of some of the unit cells 3. In particular, the elongate members 13 are arranged in a pattern comprising an array of adjacent loops with each loop extending around a group of two or three unit cells 3, although the loops could equally extend around a single unit cell 3 or larger groups of unit cells 3.

This first type of frame 12 has particular advantages. As the elongate members 13 extend along longitudinal edge folds 4 and transverse edge folds 7, the frame 12 is easily folded together with the sheet 2 whilst still providing reinforcement. This advantage could be achieved with alternative patterns of the frame 12 in which the elongate members 13 extend along any of the folds 4 to 7. Also, the pattern of the frame 12 comprising an array of adjacent loops provides a high degree of reinforcement due to the honeycomb-like nature of the pattern.

However, it is not essential that the elongate members 13 extend along any of the folds in the pattern of folds. The elongate members 13 may alternatively extend around the tubular shape of the sheet 2 without lying along any of the folds in the pattern of folds. The second type of frame 12 shown in FIG. 45 is an example of this.

In the second type of frame 12 shown in FIG. 45, the elongate members 13 are arranged in a line extend helically around the tubular shape of the sheet 2 in the folded state. Thus in this case the elongate members 13 do not extend along any of the folds 4 to 7 but extend along the planar portions of the sheet 2 between the folds 4 to 7. The second type of frame 12 has the particular advantage of providing a high degree of reinforcement with a simple frame 12 of relatively small total extent.

Of course the second type of frame 12 shown in FIG. 45 is merely an example and in general the elongate members 13 may extend around the tubular shape of the sheet 2 in a variety of other patterns without lying along any of the folds in the pattern of folds.

The elongate members 13 can have a number of alternative forms, some examples of which will now be given.

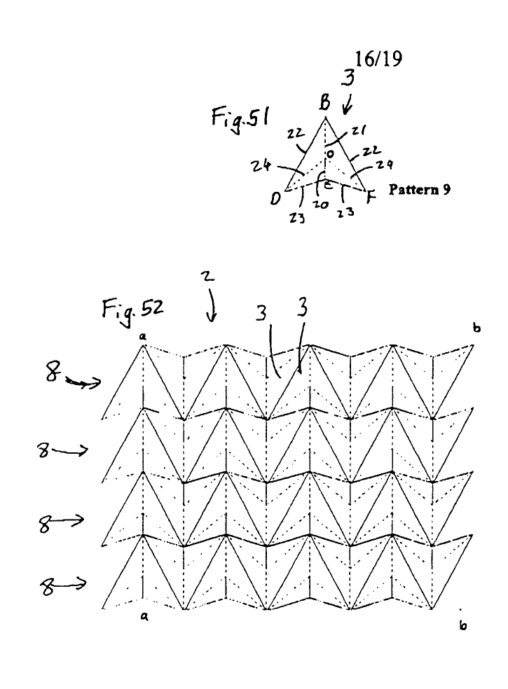

A first alternative is that the frame 12 is a separate element from the sheet 2. One example of this is that the elongate members 13 comprise wire, as shown for example in FIG. 46. Another example of this is that the elongate members 13 are formed as respective portions of a piece of sheet material, as shown for example in FIG. 47.

When the frame 12 is a separate element from the sheet 2 it may be fixed to the sheet 2, for example by an adhesive or by a physical bond of some type. However, such fixing is not essential as the frame 12 and the sheet 2 may be held together merely by friction, the folded nature of the frame 12 and the sheet 2 assisting in holding them together. The frame 12 may be arranged inside the sheet 2 to assist in holding the sheet 2 and the frame 12 together, particularly as the flexibility of the sheet 2 increases. Another possibility is that the sheet 2 is a material which is bonded directly to the frame 12, for example by being a material deposited on the frame 12 in a liquid phase and subsequently being solidified, for example by curing.

A second alternative is that the elongate members 13 are formed by portions of the sheet 2 having a thickness greater than the remaining portions of the sheet 2, as shown for example in FIG. 48.

The sheet 2 and the frame 12 (if provided) are both made of biocompatible material. Any biocompatible materials may be used. The material of the sheet 2 and the material of the frame 12 (if provided) are chosen to provide the desired physical properties for use of the stent 1 at a chosen anatomical site. The material(s) should be selected to be sufficiently rigid to hold the shape of the stent 1 between the folds 4 to 7 when implanted in a lumen. This is to perform the basic function of holding the lumen open. This must be balanced against the ease of folding the stent 1 and the need for the collapsed stent 1 to be sufficiently flexible to allow delivery to the blocked site.

A particular advantage of the use of the frame 12 is that the overall stiffness of the stent 1 is derived from both the sheet 2 and the frame 12, not solely from the sheet 2 which would otherwise reduce the choice of materials for the sheet 1. One possibility is for substantially all the desired stiffness of the stent 1 to be derived from the frame 12 in which case the sheet 2 has a high degree of flexibility. Another possibility is for the sheet 2 and the frame 12 to provide comparable degrees of stiffness.

The sheet 2 and/or the frame 12 (if provided) may be used as a carrier for a drug, in which case the sheet 2 and/or the frame 12 may be made from a material which facilitates this.

Suitable materials for the sheet 2 and the frame 12 (if provided) include a metal such as stainless steel or a shape memory alloy such as Nitinol. In the latter case, the shape memory properties may be used to assist in expansion of the stent 1 during deployment. However, the sheet 2 may be a material having a higher degree of flexibility than the material of the frame 12.

The sheet 2 may be a material of the type commonly used in covered stents, but due to the compact nature of the folding of the sheet 2 it is possible to use materials which compared to covers in existing covered stents are thicker and therefore more resistant to rupture. For example, many polymers, eg PTFE, are suitable. The sheet 2 may be a ceramic-based polymer, which is preferably elastic and non-thrombogenic.

One possiblility is that the sheet 12 comprises a nanocomposite (NC), for example an amphiphilic nanocomposite. One example is a material in which polyhedral oligomeric silsesquioxane (POSS) NC is incorporated into poly(carbonate-urea)urethane (PCU), for example as disclosed in WO-2005/070998. Such a material may provide good biostability and durability.

The material of the sheet 12 may also be one of the other materials using an NC as a base technology which are currently being developed for biomedical applications, for example a nitric oxide eluting NC or an NC having a “stem cell anchor”.

A particular advantage of the use of the frame 12 is that the sheet 2 may be made of a cheaper material than the frame 12, bringing down the cost of the stent 1. For example, the advantages a shape memory alloy such as Nitinol may be achieved without needing to make the sheet 2 from Nitonol which is expensive in sheet form, but instead making just the frame 12 from Nitonol, particularly in the form of wire in which form Nitonol is relatively cheap.

The sheet 2 is desirably selected so that the outer surface of the sheet 2 on the outside of the tubular shape of the sheet 2 provides a sufficient degree of friction to provide anchorage at the anatomical site where it is to be implanted. This may be achieved by selecting a material providing an appropriate degree of friction or by roughening the outer surface.

The sheet 2 may be made of a single material or may be a multi-layer material. In the latter case, the inner and outer layers may be selected to provide appropriate degrees of friction. Desirably the outer surface of the sheet 2 on the outside of the tubular shape of the sheet 2 provides a higher degree of friction than the inner surface of the sheet 2 of the inner side of the tubular shape of the sheet 2.

In another form, the sheet 2 may have a coating of a biocompatible material. For example, the sheet 2 may comprise a metal such as stainless steel or a shape memory alloy such as Nitinol coated by an NC of the type described above. Coating may be achieved using electrohydrodynamic spray deposition.

The stents described above may be combined together to form a larger product or may have additional components added thereto.

The dimensions of the sheet 2, the type of pattern of folds and the dimensions of the unit cell 3 within the pattern of folds are selected based on the site at which the stent is intended to be used. The stent 1 may be used for treatment at sites in any type of lumen in the body simply by choice the dimensions and mechanical properties of the sheet of the stent 1. Once deployed, the stent 1 prevents restenosis, because it is formed from a sheet 2 which is effectively continuous. The stent 1 is particularly advantageous for use in the oesophagus where restenosis is a particular problem.

The stent 1 is used in the same manner as known stents, that is by initially collapsing the stent 1 to deliver the stent 1 to the site to be treated and subsequently expanding the stent 1. Manipulation of the stent 1 is performed using conventional medical techniques.

A potential problem with the stent 1 as described above is that high stresses are developed at the nodes where the folds 4 to 7 intersect. Such stresses could create weakness at the nodes, potentially causing sheet 2 to puncture or rip. To avoid this problem, apertures 10 may be formed in the sheet 2 at the nodes where the folds intersect, or at least at those nodes where high stresses are likely to be developed.

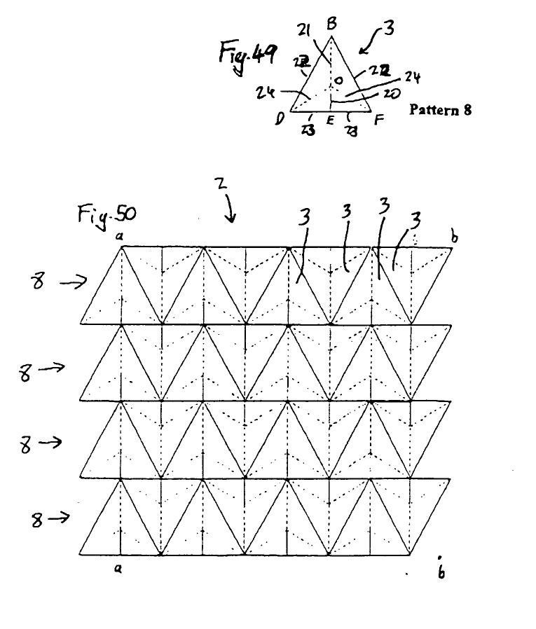

An example of such an aperture 10 formed in a sheet 2 at the node where the longitudinal edge folds 4, the transverse edge folds 5 and the angular fold 7 intersect is shown in FIG. 43. The aperture 10 in FIG. 43 is shown as being circular, but may be of any shape. The aperture 10 has a width which is greater than the width of the folds 4 to 7. The aperture 10 is sufficiently small that it does not allow significant in-growth through the aperture 10, hence effectively retaining the continuous nature of the sheet 2.

Manufacture of a stent 1 will now be described.

First, formation of the biocompatible sheet 2 will be described.

In the case that a frame 12 is provided in which the elongate members 13 of the frame 12 are portions of the sheet 2, the sheet 2 is formed with the elongate members 13 in the desired positions, for example by molding the sheet 2.

The sheet 2 may initially be planar, in which case opposed edges of the sheet 2 are subsequently joined together to form a tubular shape. In this case, in the drawings, the lines a-a and b-b may represent edges of the sheet 2 which are joined together.

Alternatively, the sheet 2 may be manufactured be formed with a tubular shape ab initio, that is with the sheet being continuous around the tubular shape. In this case, in the drawing, the lines a-a and b-b are the same imaginary line along the length of the tubular shape of the sheet 2. This latter alternative has the advantage of avoiding the need to join the edges of a planar sheet but makes it harder to form the folds.

The sheet 2 is folded with the desired pattern of folds.

Folding may be facilitated by initially forming fold lines which facilitate subsequent folding.

The fold lines may be formed by a mechanical process. One example is to score the sheet 2 mechanically. Another example is to impress the fold lines on the sheet 2, for example by a stamping or a rolling process. In that case, it is possible to impress the sheet 2 between opposed stamps or rolls having ridges along the fold lines, the stamps or rolls on one side of the sheet 2 having the pattern of hill folds and the stamps or rolls on the other side of the sheet 2 having the pattern of valley folds.

Other techniques to form fold lines are laser lithography and chemical etching.

In the case of laser lithography, a laser is used to form scores in the surface of the sheet 2 along the fold lines. The laser equipment for such processing is in itself conventional.

In the case of chemical etching, the sheet 2 is first masked by a material resistant to a chemical enchant, except along the desired pattern of folds. Then the etchant is applied to the sheet to etch scores in the pattern of folds where the masking material is not present. Subsequently the masking material is removed. Such a chemical etching process in itself is conventional. Preferably, a conventional photolithographic technique is used. In such a case, the masking material is a positive or negative photoresist applied across the entire sheet. Ultra-violet light is applied in an image of the pattern of folds, being positive or negative image for the case of a positive or negative photoresist, respectively. This alters the photoresist allowing it to be removed by the etchant in the pattern of folds, but leaving it resistant to the etchant elsewhere.

In general, the etchant and the masking material may be chosen having regard to the material of the sheet 2. However, particular possibilities are as follows.

In the case of a chemical etching of a sheet 2 of stainless steel, one possibility is to use the negative etching technique commonly used for etching stainless steel, for example using ferric chloride and 1% HCl as the etchant and using a dry film as a negative photoresist.

In the case of chemical etching of a sheet 2 of shape memory alloy, the following positive etching technique has been applied using a positive photoresist layer of solid contented HRP 504 or 506 as the masking material and using a mixture of hydrofluoric and nitric acid as the etchant. The etching method was applied to a sheet 2 of thickness 80 μm and size 80 mm×80 mm which was cleaned to improve the adhesion of the masking material. The masking material was applied by dip coating at a speed of 6 mm/min to create a coating of thickness 12 μm. The sheet 2 was then soft-baked at 75° for 30 minutes. The masking material was then exposed by UV light with a positive image of the pattern of folds on both sides of the sheet, and the image developed using PLSI: H₂O=1:4. Finally the sheet 2 was hard-baked at 120° for 60 minutes. The sheet 2 was then etched using a mixture of hydrofluoric acid and nitric acid in proportions HF 10%, HNO₃ 40%, H₂O 50% or HF:HNO₃:H₂O=1:1:2 or 1:1:4.

Other ways to chemically etch a sheet 2 of shape memory alloy are negative etching with a rubber-type of photoresist or electrochemical etching with H₂SO4/CH₃OH, for example as disclosed in Eiji Makino, et al., “Electrochemical Photoetching of Rolled Shape Memory Alloy Sheets for Microactuators”, Vol. 49, No. 8, 1998; and D M Allen, “The Principles and Practice of Photochemical Machining and Photoetching”, Adam Hilger, 1986.

In the case of a sheet 2 which is initially planar, after folding the edges of the folded sheet 2 are joined together to form the sheet 2 into a tubular shape.

As the sheet 2 is simply folded into the desired pattern, the folding process is relatively cheap.

In the case that the frame 12 is provided and that the frame 12 and the sheet 2 are separate elements, the frame 12 and the sheet 2 may be manufactured separately and attached together, and the following considerations apply.

In the case that the elongate members 13 of the frame 12 are made from wire, the frame 12 may be constructed using similar techniques to those used to form existing covered stents, although the stent 1 has the advantage that the frame 12 may in general be less complex than in existing covered stents due to the folding of the sheet 2. In the case that the elongate members 13 of the frame 12 are respective portions of a piece of sheet material, the frame 12 may be made by cutting it out from a larger piece of sheet material, for example by etching or laser cutting. The frame 12 may be cut from a sheet which is planar and formed into a tubular shape after cutting. Alternatively, a sheet already in the form of a tube may be cut to form the frame 12 with a tubular shape. To assist in folding, the frame 12 may also be formed with fold lines between the elongate members 13 using similar techniques to those described above for the sheet 2.

The frame 12 may be assembled with the sheet 2 after folding of the sheet 2.

An alternative is to attach the sheet 2 to the frame 12 before folding the sheet 2.

Another approach to manufacture is to form the sheet 2 by depositing the material of the sheet 2 on the frame 12 in a liquid phase and subsequently solidified, for example by curing. In this case, the material of the sheet 2 may be a curable resin. The sheet 2 may be deposited on the frame 12 in sheet form and then the sheet 2 and frame 12 formed into a tubular shape. In this case the deposition of the material of the sheet 2 may be performed on a flat surface. Alternatively, the sheet 2 may be deposited on the frame 12 already in a tubular shape. In this case, the material of the sheet 2 may be deposited centrifugally by introducing the material inside the frame 12 under rotation.

1. A stent comprising a biocompatible sheet having a tubular shape and being folded with a pattern of folds allowing the sheet to be collapsed for deployment of the stent, the folds being of two types, the first type being one of a hill fold and a valley fold, and the second type being the other of a hill fold and a valley fold, the pattern of folds comprising a unit cell repeated over at least a portion of the sheet, the unit cell comprising:
two longitudinal folds extending away from a common point along the tubular shape of the sheet, the first longitudinal fold being of the first type and the second longitudinal fold being of the second type;
an outer circumferential ring of four edge folds of the first type, comprising, on each side of the longitudinal folds, a minor edge fold extending from the outer end of the first longitudinal fold and a major edge fold extending from the outer end of the second longitudinal fold, the outer ends of the minor edge fold and the major edge fold on the same side of the longitudinal folds intersecting one another; and
two angular folds of the second type, each extending from the intersection of a major edge fold with a minor edge fold to the common point from which the longitudinal folds extend.

2. A stent according to claim 1, wherein the minor edge folds are collinear.

3. A stent according to claim 1, wherein the respective angles between the minor edge folds and the first longitudinal fold inside the unit cell are acute.

4. A stent according to claim 1, wherein the respective angles between the first longitudinal fold and the minor edge folds inside the unit cell are obtuse.

5. A stent according to claim 1, wherein the unit cell further comprises a ring of inner folds of the second type inside an edge fold between an angular fold and longitudinal fold, the pattern of folds inside the ring of inner folds of said second type reversing from folds of said first type to folds of said second type and vice versa.

6. A stent according to claim 1, wherein the unit cell is symmetrical about the central longitudinal fold.

7. A stent according to claim 1, wherein the folds of said first type are hill folds and the folds of said second type are valley folds.

8. A stent according to claim 1, wherein the pattern of folds comprises a unit cell of identical shape repeated over the entire sheet.

9. A stent according to claim 1, wherein the pattern of folds comprises at least one row of unit cells extending around the tubular shape of the sheet, successive unit cells in the at least one row being oriented in alternate directions along the tubular shape of the stent.

10. A stent according to claim 9, wherein the pattern of folds comprises a plurality of rows of unit cells extending around the tubular shape of the sheet, the unit cells of adjacent rows being aligned with each other such that the first longitudinal folds of unit cells in a given row meet with the second longitudinal folds of unit cells in an adjacent row.

11. A stent according to claim 9, wherein the pattern of folds comprises a single row of unit cells extending helically around the tubular shape of the sheet, the unit cells of adjacent helical turns of the row being aligned with each other such that the first longitudinal folds of unit cells in a given helical turn of the row meet with the second longitudinal folds of unit cells in an adjacent helical turn of the row.

12. A stent according to claim 1, wherein at at least some of the nodes where folds intersect the sheet has apertures having a greater width than the width of the folds.

13. A stent according to claim 1, wherein the outer surface of the sheet on the outer side of the tubular shape of the sheet has a higher degree of friction than the inner surface of the sheet on the inner side of the tubular shape of the sheet.

14. A stent according to claim 1, wherein the outer surface of the sheet on the outer side of the sheet is roughened.

15. A stent according to claim 1, wherein the outer surface of the sheet on the outer side of the sheet has a sufficient degree of friction to provide anchorage at an anatomical site.

16. A stent according to claim 1, wherein the sheet has edges joined along the length of the tubular shape of the sheet.

17. A stent according to claim 1, wherein the sheet is continuous around the tubular shape of the sheet.
